(19) United States
(12) Patent Application Publication
(10) Pub. No.: US 2012/0123376 A1
(43) Pub. Date: May 17, 2012

(54) FREEDOM-DISPOSABLE UNDERWEAR SYSTEMS FOR WOMEN AND INCONTINENT INDIVIDUALS

(76) Inventor: Maria Alves, Narberth, PA (US)

(21) Appl. No.: 13/296,684

(22) Filed: Nov. 15, 2011

Related U.S. Application Data
(60) Provisional application No. 61/414,568, filed on Nov. 17, 2010.

(51) Int. Cl. A61F 13/49; A61F 13/496; A61F 13/511

(52) U.S. Cl. 604/378; 604/385.01

(57) ABSTRACT

A women's disposable underwear system to provide discreet absorption for a sustained duration of menstruation and/or incontinence is disclosed. The disposable underwear employs fabric panels with three edges: a waist edge, a first leg edge, and a second leg edge. Disposed opposed to each other, these panels form the disposable underwear when they are seam-attached. A central panel located between the fabric panels houses an absorbent pad that wicks away moisture from the wearer of the disposable underwear. The absorbent pad may accommodate a light, medium, or heavy flow. The single-use disposable underwear may be manufactured with an uninterrupted waistband or with side fasteners to accommodate one or two side openings. Disposable underwear is manufacturable in various styles and colors.

### CROSS-REFERENCE TO RELATED APPLICATION

The present application is related to and claims priority from prior provisional application Ser. No. 61/414,568, filed Nov. 17, 2010 which application is incorporated herein by reference.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever. 37 CFR 1.71(d).

### BACKGROUND OF THE INVENTION

The following includes information that may be useful in understanding the present invention(s). It is not an admission that any of the information provided herein is prior art, or material, to the presently described or claimed inventions, or that any publication or document that is specifically or implicitly referenced is prior art.

### 1. FIELD OF THE INVENTION

The present invention relates generally to the field of disposable underwear and more specifically relates to women's disposable underwear for absorption and disposal of waste during periods of menstruation and incontinence.

### 2. DESCRIPTION OF THE RELATED ART

In modern society, a majority of individuals wear undergarments. Women experience menstrual cycles that are often less than pleasant. In fact, commiserating amongst women over this very natural function of the female reproductive system is commonplace. From puberty onward, women are resigned to combat this monthly condition for much of their lives. Whether it be the physical discomfort of the cycle itself, or the lack of comfortable options available for dealing with it, menstruation is a dreaded time for most. As such, when that “time of the month” does arrive, women are given two basic options: tampons or sanitary napkins.

Sanitary napkins are disposable absorbent pads affixed to a user's undergarments via adhesive means integrated in the underside of the pad. These sanitary napkins are bulky and uncomfortable besides often being conspicuous through one's clothing. Wearing a form fitting outfit during one's period is rarely an option when the user chooses to wear a pad. Sanitary napkins shift as a woman walks and frequently flip upside down, reversing the adhesive side upward onto the wearer, requiring constant repositioning, (not to mention drawing even further attention to an already tenuous situation). Despite the painful prospect of a pad's adhesive coating sticking to its wearer, there is also the sweaty, itchy chafing sensation that results from wearing a pad for any extended period of time (particularly in warmer weather). Developing rashes and other irritations are not uncommon for many women who choose to wear sanitary napkins. Physical activity of any kind while wearing a sanitary napkin is often an arduous and exasperating task.

The other option besides a sanitary pad is a tampon; an absorbent plug inserted directly into a woman's body. For many, inserting a tampon is uncomfortable and in some cases, painful. The risks associated with tampons, such as Toxic Shock Syndrome (T.S.S.) and other bacterial infections, have left an uneasy feeling in many female consumers, who shun the merits of tampons despite their convenience. Those who do choose to wear tampons change them rather frequently, as fear of T.S.S. and other infections is an ever present concern for most modern women. Feeling ambivalent about either choice is the norm, and yet alternative options are hard to find. Some alternatives currently available on the market are reusable wash pads, sea sponges, and “menstrual cups”, all of which are inconvenient and unrealistic for various reasons.

Another issue most women face is sacrificing certain underwear during their period. Many women, in an effort to preserve their underwear, will designate certain pairs to that time. Ruining underwear is only part of the humiliation suffered by many females during their monthly cycles. Oftentimes blood stains make their way through the pad and underwear and onto a woman's clothes. In terms of embarrassment, no woman can deny the sheer honor of discovering a blood stain on one's clothes and the frantic scramble to conceal it before others find out. Putting the embarrassment and humiliation aside, the cost factor of having to replace clothing as a result of staining adds insult to injury. A blood stain that has set into a fabric for a good period of time has almost no chance of coming out. And so, the unfortunate truth seems to be that discomfort, embarrassment and self-consciousness are all overwhelming themes throughout many females' menstrual cycles.

Various attempts have been made to solve the above-mentioned problems such as those found in U.S. Pat. and Pub. Nos. 2009/0240226; 4,560,381; 2010/0318051; 7,722,593; 2008/0140041; 6,231,558; 6,367,089; and 7,240,375. This prior art is representative of disposable underwear. None of the above inventions and patents, taken either singly or in combination, is seen to describe the invention as claimed.

Ideally, a women's disposable underwear for absorption and disposal of waste during periods of menstruation and incontinence should be comfortable, absorbent, secure, stylish and, yet, would operate reliably and be manufactured at a modest expense. Thus, a need exists for a reliable women's disposable underwear system to provide discreet absorption for a sustained duration of menstruation and/or incontinence and thereby increase the comfort, security, and confidence of a user and to avoid the above-mentioned problems.

### BRIEF SUMMARY OF THE INVENTION

In view of the foregoing disadvantages inherent in the known disposable underwear art, the present invention provides a novel Disposable Underwear Systems for Women and Incontinent Individuals system. The general purpose of the present invention, which will be described subsequently in greater detail, is to provide a comfortable and discreet absorption means for a sustained duration of menstruation and/or incontinence.

A disposable underwear system is disclosed herein preferably composed of disposable underwear for use by females and incontinent individuals. This disposable underwear is designed to be constructed using outer and inner fabric panels. In preferred embodiments, the outer fabric panel has three outer edges: a waist outer edge, a first leg outer edge, and a second leg outer edge. Also in preferred embodiments, the inner fabric panel has three inner-outer edges: a waist inner-outer edge, a first leg inner-outer edge, and a second leg inner-outer edge. The outer and inner fabric panels mirror each other in pattern form and may be seam-attached about the three outer edges and the three inner-outer edges. As the outer and inner fabric panels are disposed opposite each other, seam-attaching these panels results in the formation of disposable underwear.

The disposable underwear preferably includes a central panel located between the outer and inner fabric panels. This central panel is itself absorbent, and, in preferred embodiments, it includes an additional built-in absorbent pad. Made up of microfiber material and covered in a fleece surface, the absorbent pad provides added comfort and protection against leaks. The absorbent pad may maintain a light, medium, or heavy menstrual flow absorbency threshold and wicks moisture away from a wearer's body. Similarly, the absorbent material of the central panel may act as a secondary moisture wick for increased comfort and security. To increase the aesthetic characteristics of the disposable underwear, the built-in absorbent pad is designed for manufacture in colors matching those of the fabric panels surrounding it.

Used by women during a menstrual period, disposable underwear is designed to be discarded after one absorbent use. As replacing soiled disposable underwear is necessary at various points throughout a menstrual period, ease of removing and donning disposable underwear is paramount. One embodiment of disposable underwear is that of a solid-piece construction (requiring a wearer's legs be inserted through the first and second leg holes). Alternatively, one or two side-access openings may be employed for removing and replacing disposable underwear without completely removing a lower garment such as shorts or pants. In such an embodiment, the disposable underwear may be maneuvered around the wearer's legs and fasteners may be employed as closure means to secure the garment around the wearer's waist or hips. These side-mounted fasteners may be buttons, snaps, or the like, arranged in various configurations, such as being vertically aligned. Disposable underwear may be manufactured in myriad styles such as briefs, bikinis, boy shorts, and the like in order to meet the needs and desires of consumers.

Prior to donning disposable underwear, a wearer may choose whether or not to open the side fasteners to put on the garment. The wearer may then don the garment by aligning and then inserting a first leg into the first leg opening and repeating the process with a second leg and corresponding second leg opening. The wearer may then pull up the fabric panels and secure the waist edge about the hips or waist (fastening all the necessary closure means). At the wearer's discretion, or at such time that the garment becomes soiled, the disposable underwear may be removed (by employing a reverse of the previously stated process) and discarded in a trash receptacle. Optionally, a wearer may replace the soiled garment with a fresh one and continue their scheduled daily activities.

The present invention holds significant improvements and serves as a women's disposable underwear system. For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and method(s) of use for the present invention, Disposable Underwear Systems for Women and Incontinent Individuals, constructed and operative according to the teachings of the present invention.

FIGS. 1A and 1B show perspective views illustrating a women's disposable underwear system in an in-use condition with fastening means according to an embodiment of the present invention.

FIGS. 2A and 2B are perspective views illustrating both a front (FIG. 2A) and rear (FIG. 2B) view of the women's disposable underwear for menstruating and/or incontinent individuals displaying the inner and outer fabric panels according to an embodiment of the present invention of FIGS. 1A and 1B.

FIG. 3 is a perspective view illustrating the women's disposable underwear with optional fasteners, buttons, and snaps according to an embodiment of the present invention of FIGS. 1A-2B.

FIG. 4A is a perspective view illustrating the women's disposable underwear with a single-side and double-side closure options according to an embodiment of the present invention of FIGS. 1A-3.

FIG. 4B shows a perspective view illustrating the women's disposable underwear with a central panel and a built-in absorbent pad consisting of microfiber and a fleeced surface according to an embodiment of the present invention of FIGS. 1A-4A.

FIG. 5 is a flowchart illustrating a method of use for the women's disposable underwear system according to an embodiment of the present invention of FIGS. 1A-4B.

The various embodiments of the present invention will hereinafter be described in conjunction with the appended drawings, wherein like designations denote like elements.

### DETAILED DESCRIPTION

As discussed above, embodiments of the present invention relate to a women's disposable underwear device and more particularly to a disposable underwear system for women and incontinent individuals as used to improve the comfort and absorption capacity of disposable underwear for a sustained duration of menstruation and/or incontinence.

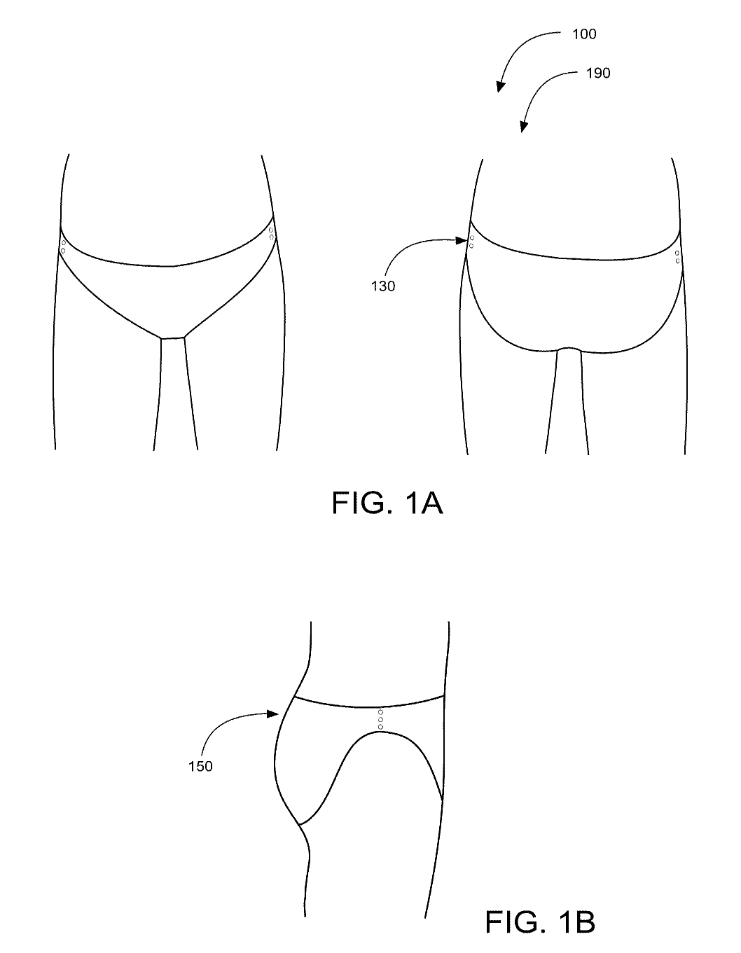

Referring to the drawings by numerals of reference there are shown in FIGS. 1A and 1B, perspective views illustrating women's disposable underwear system 100 in ‘in-use condition’ 190 with fastening means 130 according to an embodiment of the present invention.

Women's disposable underwear system 100 preferably comprises: disposable underwear 150 for use by female users having at least one outer fabric panel 120 and at least one inner fabric panel 110, at least one central panel 170, and at least one built-in absorbent pad 180. In the present embodiment, outer fabric panel 120 preferably has three outer edges comprising: first waist outer edge 121, first leg outer edge 123, and second leg outer edge 125. Similarly, inner fabric panel 110 of the present embodiment preferably has three corresponding inner-outer edges comprising: first waist inner-outer edge 111, first leg inner-outer edge 113, and second leg inner-outer edge 115.

Outer fabric panel 120 and inner fabric panel 110 may be made of any number of fabric types, such as cotton, polyester, silk, spandex, or the like. Various materials (such as cotton, polyester, silk, spandex, etc.) and/or decorative embellishments (such as lace, piping, etc.) may also be used when constructing each of the outer edges and inner-outer edges (first waist outer edge 121, first leg outer edge 123, and second leg outer edge 125; first waist inner-outer edge 111, first leg inner-outer edge 113, and second leg inner-outer edge 115) of the design. Special attention should be paid to the colors and prints of fabrics and materials used to construct outer fabric panel 120 and inner fabric panel 110 in order to appeal to wearers of disposable underwear 150. Those with ordinary skill in the art will now appreciate that upon reading this specification and by their understanding the art of garment design and construction as described herein, methods of designing and constructing disposable underwear 150 to be comfortable and aesthetically pleasing will be understood by those knowledgeable in such art.

Within the particular embodiment shown, outer fabric panel 120 and inner fabric panel 110 effectively mirror each other in pattern-form and are preferably disposed opposed to each other. Outer fabric panel 120 and inner fabric panel 110 may be seam-attached about the three outer edges: first waist outer edge 121, first leg outer edge 123, and second leg outer edge 125 and corresponding inner-outer edges: first waist inner-outer edge 111, first leg inner-outer edge 113, and second leg inner-outer edge 115 of the outer fabric panel 120 and inner fabric panel 110, respectively, thereby forming disposable underwear 150 in this combined state. Central panel 170 may be incorporated in the space between inner fabric panel 110 and outer fabric panel 120 at the midpoint between the front and back sections of disposable underwear 150. Preferably, absorbent pad 180 may be integrally affixed to central panel 170 to act as primary absorbent means of disposable underwear 150.

As primary source of absorbent means, absorbent pad 180 preferably comprises microfiber 183 and fleeced surface 185. Acting as a secondary source of absorbent means, central panel 170 is also preferably constructed of absorbent materials such as those provided for absorbent pad 180. Upon reading this specification, it should be appreciated that, under appropriate circumstances, considering such issues as user preferences, design preference, structural requirements, marketing preferences, cost, available materials, technological advances, etc., other absorbent material arrangements such as, for example, cotton, hemp, wool, bleached wood pulp, man-made absorbent materials, etc., may be sufficient.

Various attachment means such as cotton thread, polyester thread, monofilament, and the like may be used in seam-attaching outer fabric panel 120 to inner fabric panel 110, in affixing central panel 170 to outer fabric panel 120 and inner fabric panel 110, and also when attaching absorbent pad 180 to central panel 170. Those with ordinary skill in the art will now appreciate that upon reading this specification and by their understanding the art of garment design and construction as described herein, methods of joining individual garment pieces in construction of a single unit will be understood by those knowledgeable in such art.

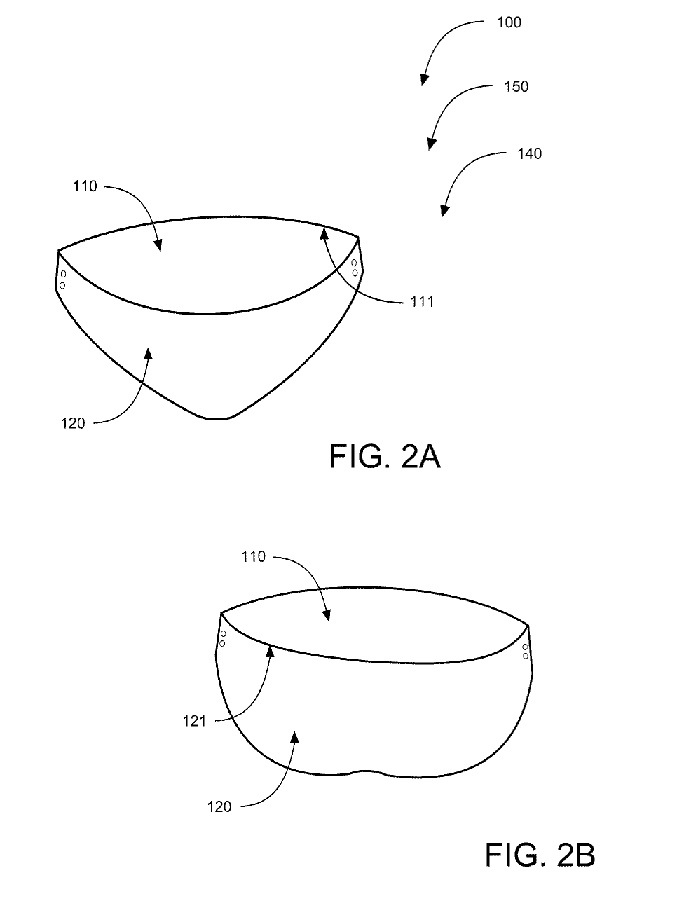

Referring now to FIGS. 2A and 2B, perspective views illustrating both a front (FIG. 2A) and rear (FIG. 2B) view of women's disposable underwear 150 for menstruating and/or incontinent individuals displaying inner fabric panel 110 and outer fabric panel 120 according to an embodiment of the present invention of FIGS. 1A and 1B.

As women's disposable underwear system 100 may be worn by women during a menstrual period or by persons affected by bouts of incontinence, it is effectively a single-use article 140 that may be discarded after one absorbent use. Shown in the present one-piece embodiment, absorbent women's disposable underwear system 100 ensures comfort in reliability and protection by effectively preventing the shifting or repositioning of absorbent pad 180 to traditional underwear. Preferably, women's disposable underwear system 100 may be manufactured in sizes similar to those currently available in brands of traditional multi-use underwear.

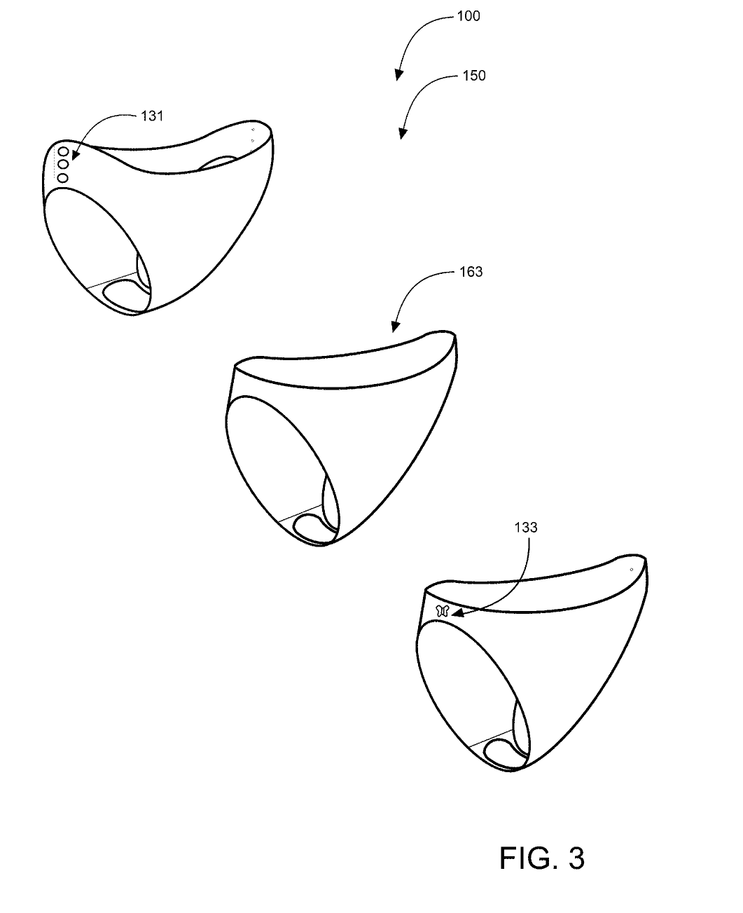

FIG. 3 shows a perspective view illustrating women's disposable underwear 150 with optional fastener(s) 131 and button(s) 133 according to an embodiment of the present invention of FIGS. 1A-2B.

Several variations for securing disposable underwear 150 to a wearer's waist are possible as shown in FIG. 3. A continuous, or uninterrupted, waistband may be employed to secure the garment using such means as an elastic band, a drawstring, or equivalent means. Conversely, the front and rear portions of disposable underwear 150 may be separated at the waistband and may be releasably joined to form a functioning disposable underwear 150 unit by side-mounted fastening means 130 (e.g., fastener 131, button 133, or snap 135 closure means). Upon reading this specification, it should be appreciated that, under appropriate circumstances, considering such issues as user preferences, design preference, structural requirements, marketing preferences, cost, available materials, technological advances, etc., other fastening and/or closure arrangements such as, for example, hook-and-eye closures, toggles, laces, etc., may be sufficient.

At least one fastener 131, button 133, snap 135, or the like may be used in variations employing fastening means 130; however, multiple fastening means 130 may be used to reinforce the integrity of the garment or to enhance the aesthetic qualities of disposable underwear 150. While the present embodiment illustrates side-mounted fastening means 130, other embodiments may utilize alternate placement of such fastening means 130 on disposable underwear 150. As shown in the present embodiment, fastening means 130 are preferably aligned vertically (other embodiments may allow for different alignment of fastening means 130, allowing for myriad variations of fastening means 130 configurations).

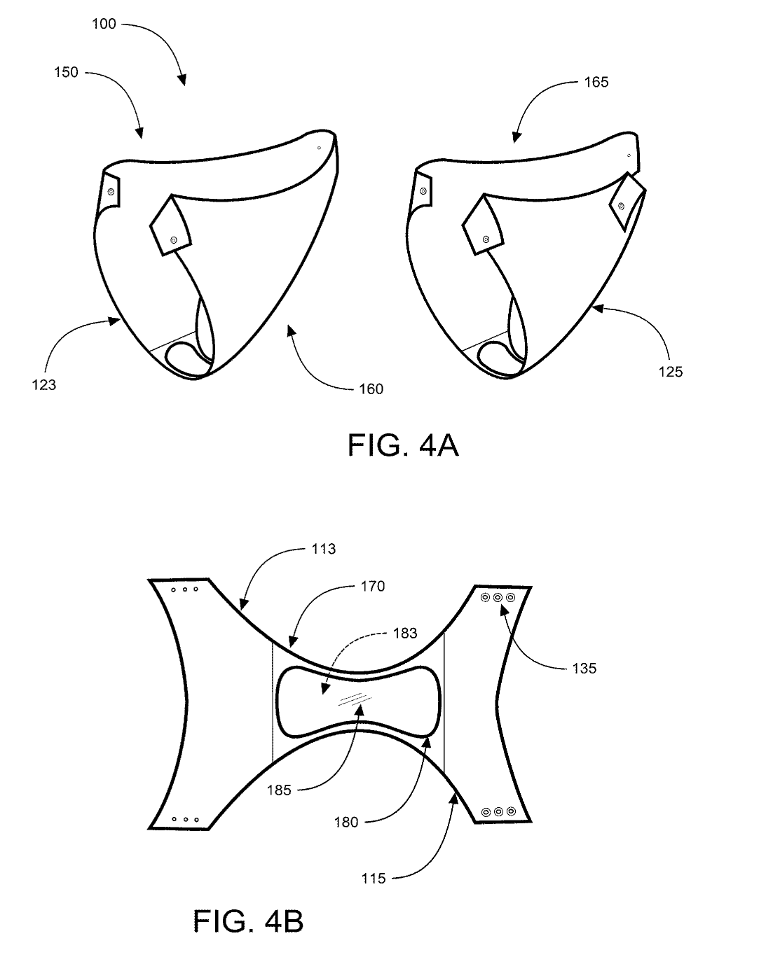

Referring now to FIG. 4A, a perspective view illustrating women's disposable underwear 150 with single-side closure 160 and double-side closure 165 options with snap(s) 135 according to an embodiment of the present invention of FIGS. 1A-3.

According to the present embodiment, side-mounted fastening means 130 allow a female wearer the option of at least three configurations for donning disposable underwear 150. Each of the fastening means 130 may be fastened (standard closure 163), fastening means 130 on one side of the garment may be fastened and the others unfastened (single-side closure 160), or all fastening means 130 on either side of disposable underwear 150 may be unfastened (double-side closure 165). A wearer may adjust fastening means 130 to a desired configuration to maximize convenience and comfort when donning disposable underwear 150. The single-side closure 160 and double-side closure 165 configurations of disposable underwear 150 allow a female wearer to remove and/or replace disposable underwear 150 without having to completely remove a lower garment such as pants or shorts.

FIG. 4B shows a perspective view illustrating women's disposable underwear 150 with central panel 170 and built-in absorbent pad 180 comprising microfiber 183 and fleeced surface 185 according to an embodiment of the present invention of FIGS. 1A-4A.

Absorbent pad 180 substantially acts as a first moisture wick for disposable underwear 150 in the present embodiment. Discharged material from menstruation and/or incontinence may be absorbed through fleeced surface 185 into microfiber 183, thereby pulling moisture away from a wearer's skin. Central panel 170 may act as a secondary moisture wick by accumulating into its absorbent surface any residual moisture from a wearer's skin not absorbed by absorbent pad 180. As flow volumes fluctuate during a menstrual period or instance of incontinence, absorbent pad 180 may accommodate the absorption of a light, medium, or heavy flow of discharge. This may be accomplished by manufacturing disposable underwear 150 with at least three absorbency threshold levels (i.e., light, medium, heavy) or by employing absorbent pad 180 with maximum absorbency threshold for any disposable underwear 150 manufactured. Regardless of its flow absorbency threshold, each absorbent pad 180 may be designed in a color matching that of the disposable underwear 150 to provide a discreet blending means for absorbent pad 180.

Women's disposable underwear system 100 may be sold as kit 440 comprising the following parts: at least one disposable underwear 150 at least one outer fabric panel 120; at least one inner fabric panel 110; at least one central panel 170; at least one absorbent pad 180; and at least one set of user instructions. Women's disposable underwear system 100 may be manufactured and provided for sale in a wide variety of sizes and shapes for a wide assortment of applications. Upon reading this specification, it should be appreciated that, under appropriate circumstances, considering such issues as design preference, user preferences, marketing preferences, cost, structural requirements, available materials, technological advances, etc., other kit contents or arrangements such as, for example, including more or less components, customized parts, different color combinations, parts may be sold separately, etc., may be sufficient.

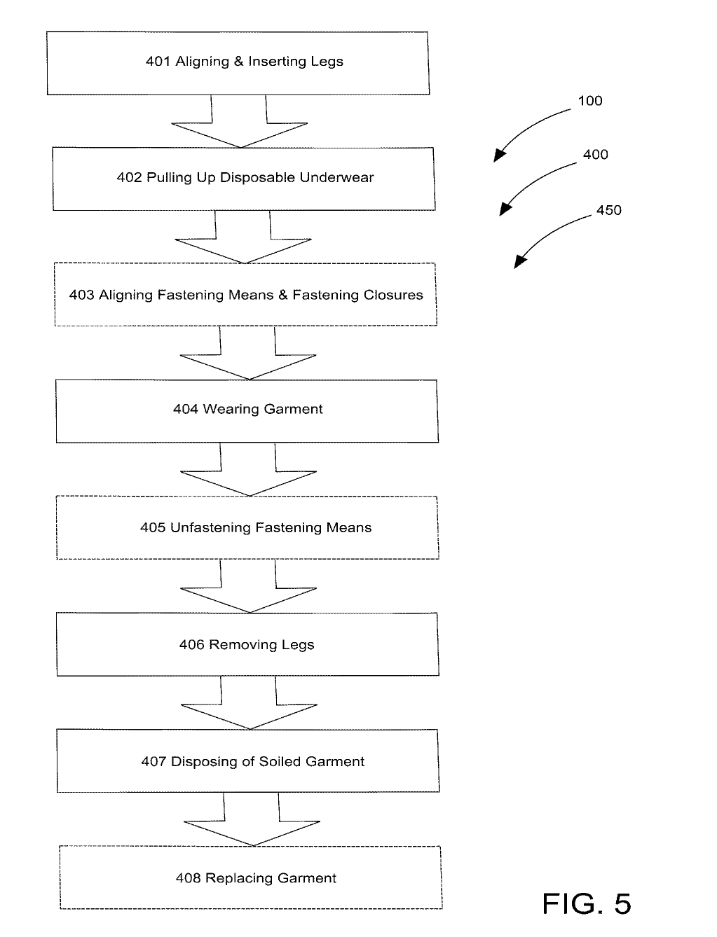

FIG. 5 is a flowchart illustrating a method of use 400 for women's disposable underwear system 100 according to an embodiment of the present invention of FIGS. 1A-4B.

A method of using (at least hereby enabling method of use 400) women's disposable underwear system 100 preferably comprises the steps of: step one 401 aligning wearer's first leg with corresponding first leg edge and wearer's second leg with corresponding second leg edge, inserting first leg into opening formed by first leg edge, and inserting second leg into opening formed by second leg edge; step two 402 pulling up disposable underwear 150 to place waist edge about a wearer's hips or waist; step three 403 aligning fastening means 130 and fastening closures; step four 404 wearing garment until such time that the garment becomes soiled; step five 405 unfastening fastening means 130; step six 406 removing first leg from opening formed by first leg edge and removing second leg from opening formed by second leg edge; step seven 407 disposing of soiled garment; step eight 408 replacing soiled garment with an unsoiled garment using step one 401, step two 402, and step three 403.

It should be noted that step three 403, step five 405, and step eight 408 are optional steps and may not be implemented in all cases. Optional steps of method 400 are illustrated using dotted lines in FIG. 5 so as to distinguish them from the other steps of method 400.

It should be noted that the steps described in the method of use can be carried out in many different orders according to user preference. Upon reading this specification, it should be appreciated that, under appropriate circumstances, considering such issues as design preference, user preferences, marketing preferences, cost, structural requirements, available materials, technological advances, etc., other methods of use arrangements such as, for example, different orders within above-mentioned list, elimination or addition of certain steps, including or excluding certain maintenance steps, etc., may be sufficient.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A disposable underwear system comprising:
disposable underwear for use by female users having,
at least one outer fabric panel having three outer edges, said three outer edges comprising a first waist outer edge, a first leg outer edge, a second leg outer edge,
at least one inner fabric panel having three inner-outer edges, said three inner-outer edges comprising a first waist inner-outer edge, a first leg inner-outer edge, a second leg inner-outer edge;
a central panel; and
a built-in absorbent pad;
wherein said outer fabric panel and said inner fabric panel are disposed opposed to each other and form said disposable underwear;
wherein said outer fabric panel and said inner fabric panel mirror each other in pattern-form;
wherein said outer fabric panel and said inner fabric panel are seam-attached about said three outer edges and said three inner-outer edges;
wherein said central panel is located between said inner fabric panel and said outer fabric panel;
wherein said central panel is absorbent; and
wherein said disposable underwear system is wearable by women during a menstrual period and are disposable after absorbent use.

2. The disposable underwear system of claim 1 wherein said built-in absorbent pad comprises microfiber.

3. The disposable underwear system of claim 2 wherein said built-in absorbent pad further comprises a fleeced surface.

4. The disposable underwear system of claim 2 wherein said built-in absorbent pad comprises a light menstrual flow absorbency threshold.

5. The disposable underwear system of claim 2 wherein said built-in absorbent pad comprises a medium menstrual flow absorbency threshold.

6. The disposable underwear system of claim 2 wherein said built-in absorbent pad comprises a heavy menstrual flow absorbency threshold.

7. The disposable underwear system of claim 2 wherein said built-in absorbent pad comprises a color matching a color of said disposable underwear.

8. The disposable underwear system of claim 1 wherein said disposable underwear comprise fasteners.

9. The disposable underwear system of claim 8 wherein said fasteners comprise side-mounted said fasteners.

10. The disposable underwear system of claim 9 wherein said side-mounted said fasteners comprise buttons.

11. The disposable underwear system of claim 10 wherein said buttons are vertically aligned.

12. The disposable underwear system of claim 10 wherein said buttons use snap closures.

13. The disposable underwear system of claim 1 wherein said disposable underwear system is useful by an incontinent individual.

14. The disposable underwear system of claim 1 wherein said disposable underwear are one-time use devices.

15. The disposable underwear system of claim 3 wherein said built-in absorbent pad is a first moisture wick.

16. The disposable underwear system of claim 1 wherein said central panel is a second moisture wick.

17. The disposable underwear system of claim 12 wherein said buttons use snap closures permit said female user to remove said disposable underwear without completely removing a lower garment.

18. The disposable underwear system of claim 12 wherein said lower garment comprises pants.

19. A disposable underwear system comprising:
disposable underwear for use by females and incontinent users having,
at least one outer fabric panel having three outer edges, said three outer edges comprising a first waist outer edge, a first leg outer edge, a second leg outer edge,
at least one inner fabric panel having three inner-outer edges, said three inner-outer edges comprising a first waist inner-outer edge, a first leg inner-outer edge, a second leg inner-outer edge;
a central panel; and
a built-in absorbent pad;
wherein said outer fabric panel and said inner fabric panel are disposed opposed to each other and form said disposable underwear;
wherein said outer fabric panel and said inner fabric panel mirror each other in pattern-form;
wherein said outer fabric panel and said inner fabric panel are seam-attached about said three outer edges and said three inner-outer edges;
wherein said central panel is located between said inner fabric panel and said outer fabric panel;
wherein said central panel is absorbent;
wherein said built-in absorbent pad comprises microfiber;
wherein said built-in absorbent pad further comprises a fleeced surface;
wherein said built-in absorbent pad comprises a light, medium or heavy menstrual flow absorbency threshold;
wherein said built-in absorbent pad is a first moisture wick;
wherein said central panel is a second moisture wick;
wherein said built-in absorbent pad comprises a color matching a color of said disposable underwear;
wherein said disposable underwear comprise side-mounted fasteners;
wherein said side-mounted fasteners comprise buttons;
wherein said buttons are vertically aligned;
wherein said buttons use snaps and snap receivers;
wherein said buttons use snap closures permit said female user to remove said disposable underwear without completely removing a lower garment;
wherein said lower garment comprises pants or shorts;
wherein said disposable underwear are one-time use devices; and
wherein said disposable underwear system is wearable by women during a menstrual period and are disposable after absorbent use.

20. A method of using a disposable underwear system comprising the steps of:
aligning wearer's first leg with corresponding first leg edge and second leg with corresponding second leg edge, inserting first leg into opening formed by said first leg edge, and inserting second leg into opening formed by said second leg edge;
pulling up disposable underwear to place waist edge about a wearer's hips or waist;
aligning fastening means and fastening closures; and
wearing garment until such time that said garment becomes soiled and then disposing of said garment.
